(19) United States
(12) Patent Application Publication
(10) Pub. No.: US 2023/0059925 A1
(43) Pub. Date: Feb. 23, 2023

(54) Macromolecular Sequencing by Quantum Transport Through Molecular Bridges

(71) Applicant: The Charles Stark Draper Laboratory, Inc., Cambridge, MA (US)

(72) Inventors: Isaac W. Moran, Maynard, MA (US); Daniel A. Valencia, Arlington, MA (US)

(21) Appl. No.: 17/889,793

(22) Filed: Aug. 17, 2022

Related U.S. Application Data
(60) Provisional application No. 63/234,574, filed on Aug. 18, 2021.

(51) Int. Cl. G01N 33/487; C12Q 1/6869; G01N 27/414; B82Y 40/00

(52) U.S. Cl. CPC G01N 33/48721; C12Q 1/6869; G01N 27/4145; B82Y 40/00

(57) ABSTRACT

A Fano resonator device can be used to sequence DNA or other macromolecules. The device includes customized molecular components, informed by computation analysis. Techniques for preparing and using the device also are disclosed. The device can be incorporated in a system that further includes a sample processing component and a flow chamber.

### RELATED APPLICATIONS

This application claims the benefit under 35 USC 119(e) of U.S. Provisional Application No. 63/234,574, filed on Aug. 18, 2021, which is incorporated herein by reference in its entirety.

### BACKGROUND OF THE INVENTION

Currently, DNA sequencing is performed using sequence-by-synthesis (SBS). Commercially, this process is performed by the NextSeq or NovaSeq instruments from Illumina, Inc. Mechanistically, SBS generates DNA sequences through an extensive process in which source DNA molecules are fragmented, the fragments are replicated and grafted to an optically readable flow cell, and each fragment is re-sequenced with optically active bases which emit a specific wavelength of light for each inclusion event. The entire re-sequencing process is recorded by a photodiode. Once the fragmented data is collected, sophisticated algorithms are used to reconstruct the original sequence.

Outside of DNA sequencing, the analysis of other biomolecules, such as proteins or carbohydrates, is much less systematic or consolidated. Essentially, no commercial instruments or technology exists to automatically sequence these or other macromolecules. The processes that are utilized involve digesting the molecules in strong acids and evaluating their fragmentation by mass spectrometry.

### SUMMARY OF THE INVENTION

Current DNA sequencing technologies, while systematic, are hindered by complex signals with low correlation to molecular structure which require extensive time (days) and computational power to analyze using predominantly bulky, environmentally sensitive tools. All examples, including sequence-by-synthesis, nanopore (Oxford Nanopore Technologies), and polymerase rely on fragile biomolecular components and expensive reagents that need cold storage. Consequently, nucleic acid sequencing remains a slow, hands-on process, where immense resources are focused on deciphering ambiguous signals and accommodating large error rates (˜3%). Further still, consolidated sequencing of proteins or other polymers is practically non-existent.

Thus, a need exists for approaches and techniques that address these deficiencies and are applicable to sequencing DNA or other macromolecules.

A need also is raised by the recognition that devices based on nanoribbons formed through lithographically (top-down) patterning graphene are hindered by re-arrangements and incur scattering defects (if a structure with a thickness (cross-section) small enough to be effective must be formed). Larger graphene ribbons, even though easier to fabricate, have relatively large cross-sections, resulting in a diminished signal (since a passing analyte molecule will not have a significant impact on the quantum states).

The present invention generally relates to quantum transport sequencing or QT-Seq approaches, aiming to meet at least some of the limitations and challenges associated with existing technologies.

Approaches described herein are realized through theoretical and computational advances combined with fabrication processes and detection techniques aimed at producing devices and methods capable of identifying single molecules, DNA for instance.

In many embodiments, the identification is based on Fano resonance, an approach in which a target molecule interacts with a ballistic conductor. Further embodiments address challenges presented by ballistic conductors based on current graphene nanoribbons. One implementation of the invention, for example, relies on molecular bridges. Such bridges can possess the same or similar ballistic conductivity as a nanographene ribbon but can be readily synthesized in solution with custom structures for both transport and e-coupling or quantum coupling. In many cases, molecular bridges can have cross-sections in the range of 1-2 nanometer (nm), providing an ideal transport cross-section for promoting Fano resonance with adsorbing molecules.

In some of its aspects, the invention addresses existing needs by combining: a) the latest capabilities in solution based graphene ribbon synthesis (molecular bridges) with b) the latest in graphene-nanogap lithography techniques and c) the observation that molecular bridge based devices demonstrate measurable Fano-resonance. Some embodiments relate to a Fano resonance device that includes a molecular bridge. Other embodiments address the production of such a device, while further embodiments relate to applications and uses of the device.

Bridge molecules can be synthesized, for instance, by aromatic cross coupling reactions. Subsequent benzannulations reactions can be conducted to generate a graphitic-like backbone. The molecules can be deposited across and coupled to nano-gaps in patterned graphene electrodes.

Devices described herein include a flow arrangement in which a buffer solution containing the macromolecule of interest is directed across the molecular bridge and studied by Fano resonance transmission. In some implementations, the device is integrated in a fluidic system that includes a flow cell, typically a nanofluidic system for streaming samples (analytes in a buffer medium, for instance) over the molecular bridges. The system may involve developing waveforms for detecting streaming samples. These waveforms may further be derived through machine learning using artificial intelligence.

In one implementation, scanning waveforms are developed to analyze targets in flow. The sequencing can be demonstrated by streaming a short ssDNA strand (e.g., 3 to 4 bases long) over molecular bridges and conducting the Fano resonance transmission analysis. In some embodiments, the system includes a sample processing component from which the sample is directed to a flow chamber, then to the sequencing device for Fano resonance analysis. On-chip sample processing can be employed in some cases.

Thus, in one aspect, the invention features a method for fabricating a sequencing device. The method comprising applying a graphene monolayer onto a substrate to form a universal gate; patterning the graphene monolayer into graphene nanoribbons separated by nanogaps; depositing metal electrodes onto the graphene nanoribbons to form a FET array; and depositing molecular bridges across the nanogaps. In many cases, the molecular bridge has a cross-section no greater than about 2 nanometers and a ballistic conductivity that is the same or substantially the same as the ballistic conductivity of a graphene nanoribbon.

Another aspect of the invention relates to a sequencing device in which a substrate supports a graphene monolayer. Also included is a molecular bridge (which, in some implementations, has a cross-section no greater than 2 nanometers and a ballistic conductivity that is the same or substantially the same as the ballistic conductivity of a graphene nanoribbon) at a nanogap defined in the graphene monolayer and electrodes for providing electron transport along the molecular bridge.

In a further aspect, the invention features a sequencing system which comprises a flow chamber and a Fano resonator sequencing device comprising a molecular bridge at a nanogap defined in a graphene monolayer. In one example, the molecular bridge has a cross-section no greater than 2 nanometers and a ballistic conductivity that is the same or substantially the same as the ballistic conductivity of a graphene nanoribbon.

Yet another aspect of the invention pertains to method for sequencing a molecule. The method includes passing a sample comprising the molecule across a molecular bridge having a cross-section no greater than 2 nanometers and a ballistic conductivity that is the same or substantially the same as the ballistic conductivity of a graphene nanoribbon; and conducting a Fano resonance transmission analysis to identify the molecule or a component thereof.

Aspects of the invention are widely applicable, providing real time knowledge, with increased sequencing rates (e.g., by a factor of 100), employing molecular scale structural analysis for reduced computational complexity and high accuracy single base calling (>99%). Equipment described herein is expected to yield highly portable, robust solutions by leveraging established nano/microlithography to produce miniature, fully synthetic devices operating free of enzymatic mechanisms or other bimolecular components. Economies of scale for microfabricated devices, the absence of expensive reagents or sophisticated ASICs (application specific integrated circuits)(to interpret ambiguous signals) are expected to greatly reduce unit costs when compared to established tools. Using a low powered ASIC version can ultimately obviate the need for sophisticated and extensive computational power to derive a sequence.

Fano resonators and systems described herein are robust, fully solid-state and rely on molecular electronics, providing a platform which could overcome the limitations of existing technology and expand sequencing capabilities into the multi-molecular realm. Current bulky, time and labor-intensive methods can be improved significantly.

In contrast to technologies based on nanoribbons through lithographically (top-down) patterning graphene, aspects of the present invention harness the agility of solution based chemistry to generate molecular bridges with graphitic backbones; these bridges are small enough to have a transport conduit that is fully perturbed by a passing molecule capable of coupling to the bridge's quantum states through Fano resonance.

Aspects of the invention greatly simplify the sequencing process. Many of the embodiments described are expected to yield single molecule sequencing, multi-molecular sequencing (i.e., sequencing more than one molecule type, in contrast to SBS which can only sequence DNA as the enzymes only replicate DNA), discrete base calling, etc., and benefit from available theoretical guidance. Density functional theory (DFT), for instance, involves mature, well tested and commercially available techniques for signal prediction and comparisons.

Compared to current state-of-the-art sequence-by-synthesis (e.g., SBS, Illumina), techniques described herein also are expected to represent a signal improvement through a massively reduced error rate by enabling the processing of single macromolecular ssDNA at one time, without the need for fragmentation and later recombination of scattered sequences. Thus, in combination with single base resolution, QT-seq also offers the possibility for non-fragmentation. (Technically, nanopore sequencing avoids fragmentation, but is hampered by multi-base resolution, reading 5 bases at a time and requires to empirically identify a unique signal from all possible combinations of 5 bases.)

The short-read length, high computational complexity and inabilities to address new genomes, to name just a few inadequacies associated with existing methods, can be replaced by practically infinite read lengths, low computational complexity and full genome capabilities.

As already noted, no commercial instrument or technology exists to automatically sequence proteins, carbohydrates or other macromolecules. Processes that are currently utilized involve digesting the molecules in strong acids and evaluating their fragmentation by mass spec. These deficiencies also can be addressed by approaches described herein.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is schematic perspective view of a device employing a graphene nanoribbon;

FIG. 2A illustrates the interaction between the graphene nanoribbon (GNR) and a DNA base, cytosine;

FIGS. 2B and 2C illustrate, respectively, the transmissive and coupled states of the cytosine-GNR system in FIG. 2A;

FIG. 3 is a plot of transmission as a function of energy illustrating a transmission spectrum by Fano resonance;

FIG. 4A is a diagram of an arrangement with a molecular bridge;

FIG. 4B is a diagram of an arrangement with a DNA base adsorbed on the molecular bridge shown in FIG. 4A;

FIG. 4C is plot of transmission as a function of energy showing a transmission spectrum of molecular bases on a molecular bridge;

FIGS. 5A and 5B depict, respectively, an amine down and an acid down orientation of leucine on a molecular bridge;

FIG. 5C is a transmission spectrum by Fano resonance identifying the orientations in FIGS. 5A and 5B;

FIG. 6A illustrates the adsorption of a carbazole group present in a polymeric compound on a molecular bridge;

FIG. 6B shows a Fano resonance transmission spectrum of the carbazole monomer;

FIG. 7 is a schematic diagram of a process for preparing and using a Fano resonator device;

FIG. 8A is a schematic diagram of a system for DNA sequencing according to one embodiment of the invention; and

FIG. 8B shows another transmission spectrum.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The invention generally relates to the study of molecules such as DNA, RNA, proteins, carbohydrates, organic polymeric compounds and so forth. In specific embodiments, the invention relates to the sequencing of such molecules using quantum transport (QT), a phenomenon that can be encountered when a nanostructure is connected to an external electrical current. Quantum transport is often modeled using Nonequilibrium Green Function-based techniques.

When device dimensions are smaller than the mean free path of electrons, the transport is described as ballistic. Ballistic conduction refers to the unimpeded flow of charge carriers, e.g., electrons, or energy-carrying particles, over relatively long distances in a material. Examples of ballistic conductors are nanographene ribbons (NGR), also known as graphene nanoribbons (GNR). Edge geometries often play an important role in NGRs and have been studied for various configurations such as, for instance, armchair or zigzag.

Some of the approaches described herein focus on identifying the bases that compose DNA, amino acids in a protein, etc., through an interaction known as Fano resonance, a type of resonant scattering phenomenon that gives rise to an asymmetric line-shape. Generally, the Fano resonance line-shape is due to interference between a background and a resonant scattering process. More specifically, the interference is between two scattering amplitudes, one due to scattering within a continuum of states (the background process) and the second due to an excitation of a discrete state (the resonant process). For the effect to take place, the energy of the resonant must be in the energy range of the continuum (background) states. Near the resonant energy, the background scattering amplitude typically varies slowly with energy, while the resonant scattering amplitude rapidly changes both in magnitude and phase. In more detail, for energies far from the resonant energy (Eyes), it is the background scattering process that dominates. Within 2Γᵣₑₛ (where Γᵣₑₛ describes the line width of the resonant energy), the phase of the resonant scattering amplitude changes by π. it is this rapid variation in phase that creates the asymmetric line-shape.

In theory, target molecules such as bases composing a DNA strand could be identified through their interaction with a ballistic conductor, a nanographene ribbon (NGR), for example. The ballistic conductor (here the nanographene ribbon) transmits electrons in discrete energy states depending on their energy levels. The electrons in those transmitted energy states have intrinsic resonance which can couple to orbitals of equivalent resonance in proximal molecules. This coupling, namely the Fano resonance, can be detected as a drop in transmission through the ballistic conductor at that energy level. Observing this interaction over an energy range generates a spectrum unique to the target molecule, allowing unambiguous characterization of its structure. It is, in effect, an absorption spectrum much like Fourier transform infrared (FTIR) spectrometry, only in the case of a Fano resonance spectrum the spot size reduces to the cross-section of a molecule, or in the case of a macromolecule, sub-molecular components, such as DNA bases or amino acids in a protein.

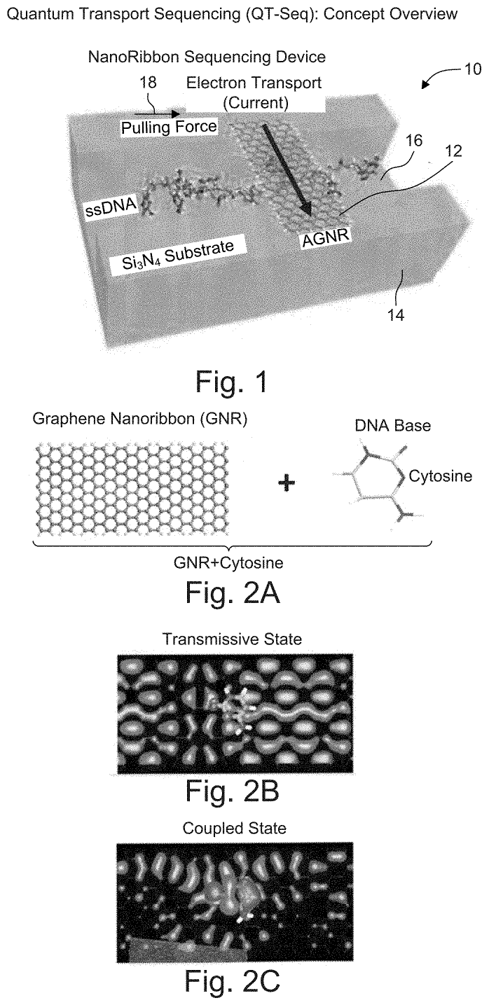

An arrangement illustrating QT-Seq principles using a graphene nanoribbon is shown in FIG. 1, in which device 10 includes a graphene nanoribbon 12, e.g., an armchair graphene nanoribbon (AGNR), supported on silicon nitride (Si₃N₄) substrate 14. Defined in the substrate is channel 16, a microfluidic or nanofluidic channel, for example. A medium, such as a buffer solution, containing a macromolecule to be analyzed, e.g., a single strand DNA (ssDNA), is driven by pulling force 18, which can be a fluid flow carrying the macromolecule. For some applications, bio-macromolecules such as ssDNA can be uncoiled to an extended configuration, by using nanoscale pillars and passing the macromolecule through an orifice which can be made of silicon nitride or another easily patterned substrate such as Si, SiOx, etc.

The electron transport (current) is indicated by the arrow perpendicular to the direction of the pulling force 18.

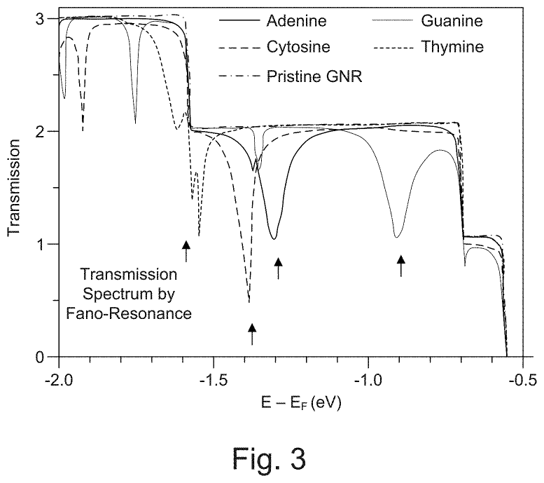

To illustrate, FIG. 2A shows the interaction of a DNA base such as cytosine with the AGNR. FIGS. 2B and 2C show, respectively, the cytosine transmissive and couple states, the two of which coexist. Transmissive states are those states that do not resonate with the imposing analyte molecule and thus show no perturbation in their transmission. (They are similar to wavelengths that are not absorbed in an FTIR spectrum). In contrast, coupled states are states able to couple through Fano resonance with the orbital energy states of the passing analyte molecule and thus are attenuated, causing a reduction in transmission (much like a band signal in an FTIR spectrum).

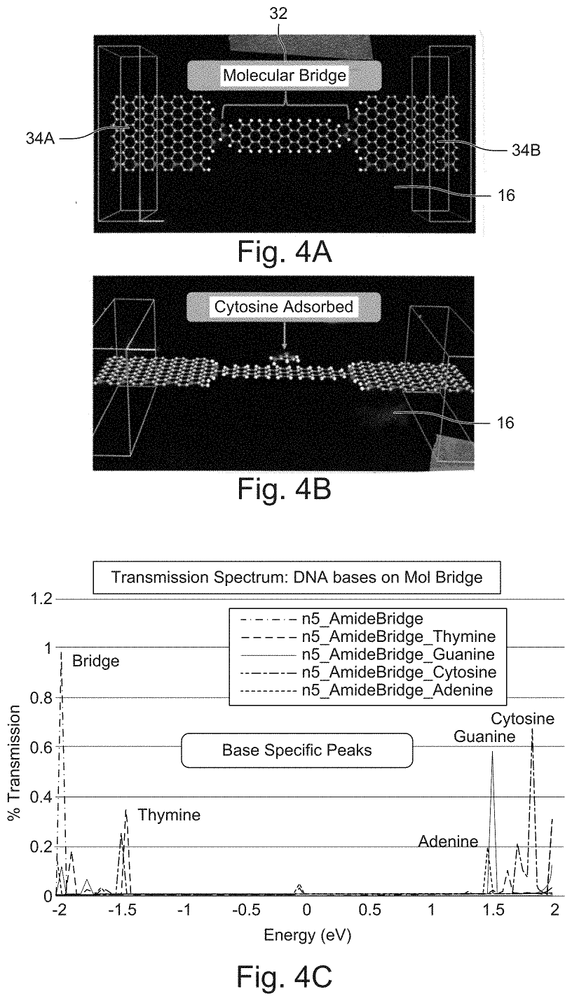

Thus, applying Fano resonance principles generates a transmission spectrum in which each of the DNA bases can be identified by a unique spectral feature, as illustrated in FIG. 3.

In the context of the analyses contemplated here, however, existing graphene nanoribbon technology may not fully meet Fano resonance requirements. For example, it is estimated that effective sequencing of DNA or proteins by Fano resonance occurs with graphene nanoribbon widths in the range of 1 nanometer (nm). Yet the current smallest seeded growth nanoribbon is considerably larger, typically about 7 nm in cross section. In addition, graphene nanoribbons are grown on substrates that may not be ideal for subsequent device fabrication.

Top down attempts to carve nano-ribbons out of graphene sheets have been attempted but the carved out ribbons undergo extensive re-arrangements of dangling bonds at the ribbon edges, imposing substantial, yet random, scattering sites and resulting in final transport properties that are highly inconsistent. Thus, there are no examples of successful or reproducible devices involving graphene nanoribbons of the size scale needed to sequence DNA or proteins by Fano resonance-based approaches.

According to some of the embodiments described herein, the limitations encountered with existing NGR technologies are addressed by molecular bridges. Nearly equivalent in structure, molecular bridges possess the same or substantially the same ballistic conductivity as a nanographene ribbon but can be readily synthesized in solution with custom structures for both transport and covalent coupling to graphitic nanogaps.

Importantly, molecular bridges typically possess very small cross-sections (typically less than 7 nm, often less than 6 nm, less than 5 nm, less than 4 nm, less than 3 nm, or less than 2 nm). In some examples, the cross section of the molecular bridge is no greater than about 2 nm, e.g., within a range of from about 1 to about 2 nm, providing a transport cross-section that is ideal for promoting Fano resonance with adsorbing molecules. Slightly larger cross section ranges (e.g., about 1 to about 3 or about 1 to about 4 nm) may work in some cases.

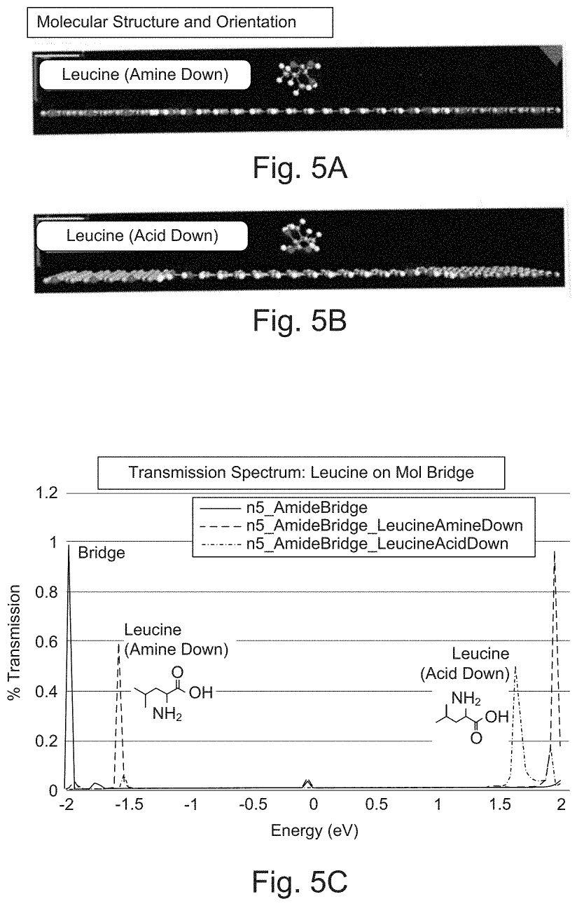

Illustrated in FIG. 4A is arrangement 30 which includes molecular bridge 32 disposed at channel 16. The bridge can couple GNR regions 34A and 34B. FIG. 4B shows a DNA base, cytosine, for instance, adsorbed by the molecular bridge. In some implementations, the molecular bridge is deposited across a nanochannel or “nanogap” formed in a graphene monolayer. The typical width of the nanogap can be within a range of from about 1 nm to about 5 nm (e.g., from about 1 to about 2, from about 1 to about 3, from about 1 to about 4 nm; or from about 2 to about 3, from about 2 to about 4, from about 2 to about 5 nm; or from about 3 to about 4, from about 3 to about 5; or from about 4 to about 5 nm). In some examples, the nanogap is no greater than about 5 nm, no greater than about 4 nm, no greater than about 3 nm, no greater than about 2 nm or no greater than about 1 nm. For electron transport, regions 34A and 34B can be provided with electrodes and such arrangements are referred to as “graphene electrodes”, e.g., nanographene electrodes. Devices described herein can include more than one channel 16, e.g., more than one nanogaps. Each gap can be fixed with a single molecular bridge of equivalent structure. Multiple parallel nanogap devices can work in concert by patterning nanogaps where each gap is separated and isolated from neighboring nanogap devices by a distance much larger than any molecule under interrogation.

Support for the use of molecular bridges was established by high level calculations performed to evaluate their potential as transport conduits capable of exhibiting Fano resonance with adsorbed molecules. The calculations relied on the non-equilibrium Green's function formalism in density functional theory (DFT-NEFG) (described in the article Fast DNA Sequencing with a Graphene-Based Nanochannel Device, by Min, S. K.; Kim, W. Y.; Cho, Y.; Kim, K. S., Nat. Nanotechnol. 2011, 6, 162-165.

Models developed in the context of the present application were based on structures similar to those for which a viable synthetic pathway is identified. Results obtained are representative of synthetically accessible devices. This stands in contrast to prior, purely theoretical work with nanographene ribbons which remains exclusively theoretical with little or no consideration for its translation to practice.

The transport calculations performed on molecular bridge structures yielded results indicating Fano resonance was active, with each DNA base producing a unique transport spectrum, as plotted in FIG. 4C, presenting transport spectra for G, A, T, and C. These spectra are clearly distinguished from each other and from the spectral features associated with the molecular bridge itself.

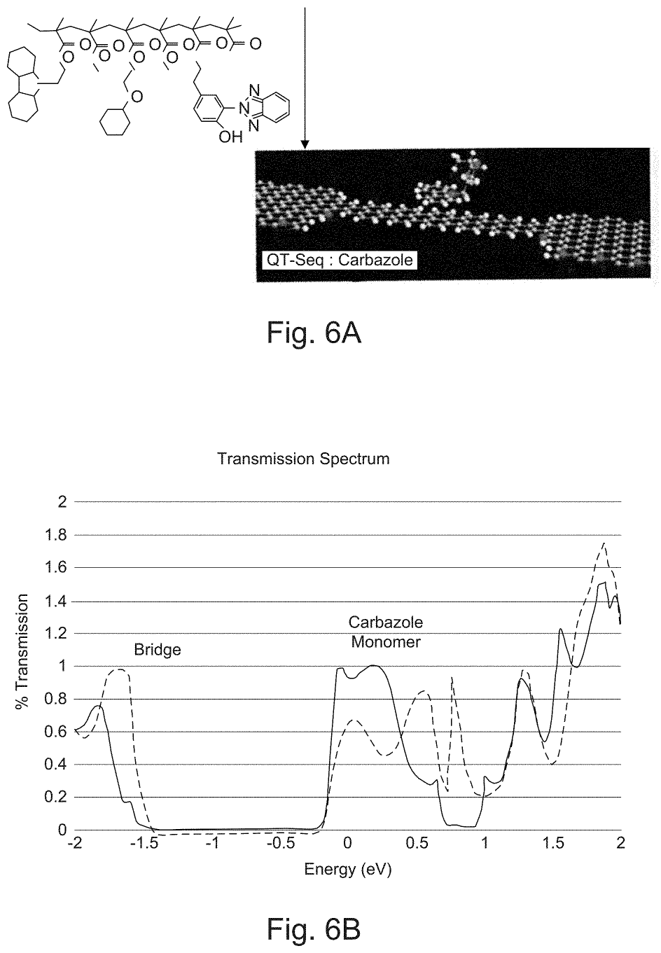

To demonstrate the potential QT-Seq for multi-molecular modeling, calculations were performed on a model interrogating the amino acid leucine. As with the DNA bases, the model yielded a distinct spectrum for leucine, a result which may be extrapolated to sequencing peptides and proteins. It was even possible to identify two orientations of the leucine molecule relative to the molecular bridge: (a) amine down (FIG. 5A); and (b) acid down (FIG. 5B). As illustrated in FIG. 5C, the two orientations yielded distinct Fano resonance transmission spectra.

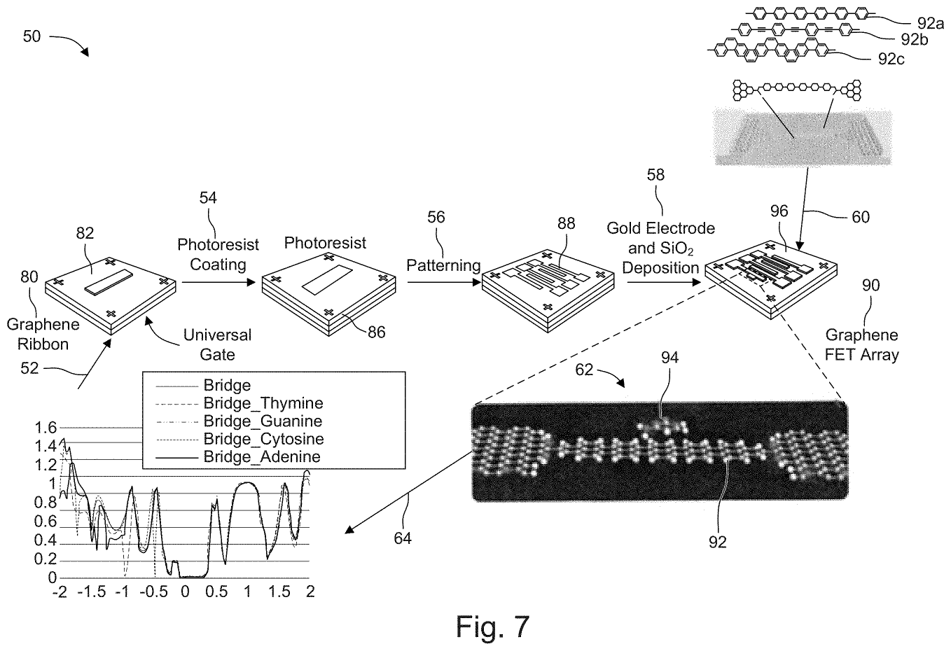

Techniques described herein also can be applied to polymeric structures such as that presented in FIG. 6A. The carbazole monomer transmission spectrum, against the spectrum of the molecular bridge, is shown in FIG. 6B.

The molecular bridge can be fabricated through a combination of organic molecular synthesis, nanopatterning of monolayer graphene, and molecular assembly. For example, bridge molecules can be synthesized by aromatic cross-coupling reactions followed by benzannulations reactions to generate graphitic-like backbone. The molecules can then be deposited across and coupled to nano-gaps in patterned graphene electrodes.

In some of its aspects, the invention relates to a Fano resonance device (also referred to herein as a Fano resonator) with customized molecular components, informed by computation analysis. Specific embodiments relate to the construction of the device, e.g., a sequencing chamber featuring molecular bridges. The initial fabrication process can involve the following phases (stages): (i) preparation of graphene nanoelectrodes on a substrate, SiOₓ/Si, for instance; (ii) synthesis of DFT-guided molecular bridges; and (iii) coupling molecular bridge structures to nanographene electrodes. A subsequent stage (iv) can be dedicated to measuring Fano resonance with DNA bases, while stage (v), which is optional, involves refining the molecular bridge structures. The completed device can then be used to sequence DNA by Fano resonance (stage (vi)).

Each stage can involve one or more steps. During stage (i), for example, a single graphene layer is transferred to a SiOₓ/Si substrate. In one implementation, nanographene electrodes are patterned on substrates composed of thermally grown silicon oxide of a sufficient thickness (˜1 μm) to insulate the overlaid graphene electrodes from the underlying, highly doped, silicon wafer, which will serve as the universal gate. The single graphene layer is then photopatterned, e.g., into isolated microscale strips, on the SiOₓ/Si substrate.

Microscale metallic (e.g., Au/Ti) electrodes, as test pad connections, are then defined over the graphene strips. The electrodes are enshrouded with an insulating layer of silicon oxide, for example, to shield them from solutions containing target molecules.

E-beam lithography in conjunction with plasma etching or e-beam defined lithography can be used to define nanogap electrodes in the graphene ribbon between the metallic electrodes.

Standard microscopy and device characterization can be undertaken to ensure that the desired nanogap and open circuit properties are achieved.

Stage (ii), namely the DFT-guided synthesis of molecular bridge structures, can involve the evaluation for optimal transmission and Fano resonant properties of an array of candidate molecular bridge structures using DFT-NEGF computational analysis. From the overall pool of structures, promising candidates can then be selected for synthesis based on computational results and synthetic accessibility. Actual synthesis can then be performed. The resulting bridge molecules can be characterized by standard analytical techniques such as nuclear magnetic resonance (NMR), mass spectrometry (MS), etc., as known in the art.

In stage (iii), molecular bridge molecules are coupled to the nanographene electrodes. The resulting bridge device can be characterized for intended structure and device properties, such as conductance and gate modulation.

The prototype device can be used to measure Fano resonance using DNA bases in stage (iv). Performing these measurements may entail constructing custom tests cells for adsorption of bases from solution. Arrangements can be configured to deliver solutions of target molecules (adenine, cytosine, thymine, and guanine) over the bridge electrodes. Typically, adsorption events are detected by monitoring known transmission states for attenuation. Upon base adsorption to a molecular bridge, Fano resonance can be evaluated by gate sweeps through pertinent energy ranges.

Optionally, the molecular bridge structures can be refined in a second round of computational studies, informed by structures derived from those that were effective in the first iteration. Thus, if Fano resonance is observed for any of the initial devices, a second array will be generated and evaluated for improved transmission and Fano resonant properties using DFT-NEGF computational analysis. The best candidates can be selected for synthesis based on computational results and synthetic accessibility. Synthesized molecules can then be characterized at the ˜100 mg scale.

The refined, second iteration molecular bridges can then be coupled to the nanographene electrodes. Structure-performance verification and Fano resonance measurements can be performed essentially as described above. Further iterations can be undertaken. Various bridge molecules obtained in any of the iterations carried out can be evaluated to select a final molecular bridge species.

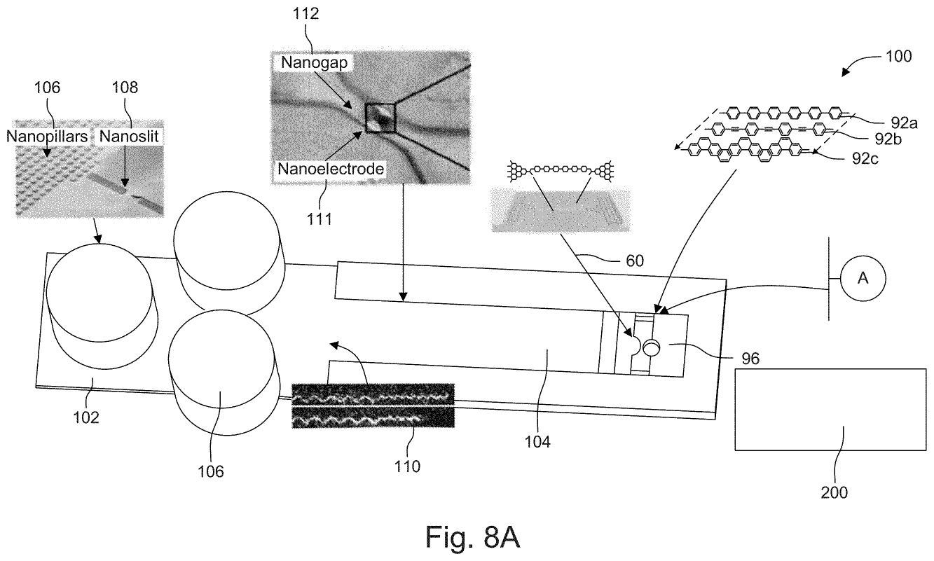

A visual depiction of steps that can be undertaken in the fabrication of the sequencing device and its use is presented in FIG. 7. As shown in this figure, overall process 50 includes step 52, in which a single graphene layer 82 is applied onto substrate 84 forming a structure that will function as the universal gate. Structure 86 is formed in step 54, involving the application of a photoresist coating. The graphene layer is patterned into graphene nanoribbons 88 in patterning step 56. Metal (e.g., gold) electrode and SiO₂ deposition are carried out in step 58 to form a graphene field-effect transistor (FET) array 90. Molecular bridges based on species 92a, 92b or 92c, generated as described above, are coupled to nanographene electrodes in step 60 to form device 96. In step 62, a target molecule 94 is adsorbed onto molecular bridge 92 (based on one of the 92a, 92b or 92c species). A Fano resonance measurement (transmission as a function of electron energy) can be obtained in step 64. In some cases, for streaming samples, for instance, scanning waveforms are developed and relied upon for target analysis. In the case of QT-seq, a waveform would involve the application of source-drain voltages applied in periodic pulses with concurrent modulation of amplitude. The periodicity of the pulse could be adjusted or optimized to probe transport through the molecular bridges during translation of an adsorbed DNA molecule under interrogation.

A sequencing chamber such as device 96 described above can be part of a system that combines sample processing, microfluidics, and sample analysis (based on Fano resonance transmission, for example). Exploiting the high performing combination of molecular bridges and coupling motifs can produce an apparatus (also referred to herein as system) aimed at sequencing a macromolecule. The entire apparatus or any components thereof can be computer-controlled for automated processing. In one implementation, the apparatus is designed for sequencing DNA. A short, single stranded DNA sequence can be used for initial evaluations.

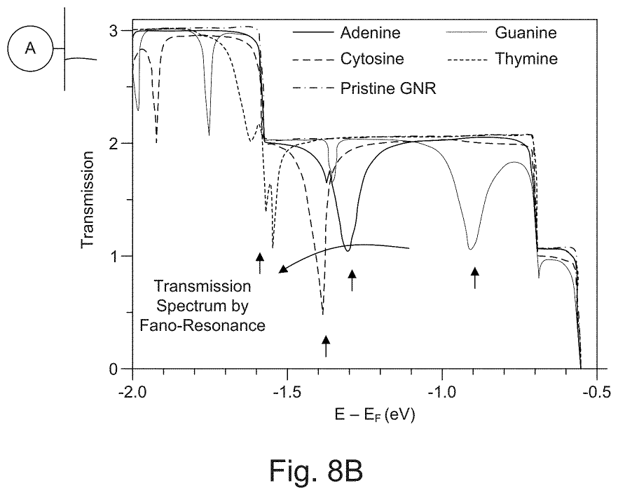

Shown in FIG. 8A, for instance, is system 100 which includes sample (e.g., DNA) processing component 102, flow chamber 104 and device 96, prepared as described above, for example.

FIG. 8B shows the transmission spectra by Fano-resonance for adenine, guanine, cytosine, thymine, and pristine GNR.

DNA processing involves the purification of a raw sample, molecular sorting and/or molecular alignment. A bio-macromolecules such as a ssDNA can be uncoiled to an extended conformation by using nanoscale pillars 106 and by passing the macromolecule through an orifice such as nano-slit 108. The slit can be formed from silicon nitride or another easily patterned substrate such as Si, SiOₓ, etc. On-chip sample processing technology can be used in some cases.

Flow chamber 104 can include at least one nanochannel 110. Nanochannel 110 can be formed by standard high resolution photolithography (deep-UV, X-ray) or e-beam lithography, for example, and can be optimized for single molecule flow to direct the target analyte to one of the molecular bridges 92 (e.g., a n5_bibenzyl bridge) of device 96. The flow can be narrowed using nanoelectrodes 111 and nanogap 112. Techniques that can be employed to prepare and operate flow chamber 104 are described in ACS Nano 2015, 9, 2, 1206-1218, https://doi.org/10.1021/nn507350e. In one example, standard high resolution photolithography for patterning is followed by aligned lamination using high resolution assembler for assembly and sealing.

System 100 can be miniaturized, to a footprint of about 3000 μm², for instance, (e.g., 30 μm by 100 μm). In one embodiment, QuantumBioSystems chips (see, e.g., Quantum Biosystems Debuts DNA Sequencer, by Ann M. Thayer, c&en, Feb. 3, 2014 Vol 92, Issue 5) are fitted with QT-Seq molecular bridges in place of their conductance electrodes.

Controlled 200 controls at least one of the operations performed in system 100. In specific embodiments, controller 200 controls the entire DNA processing, including the sample preparation, its flow into and through the flow chamber 104 and over the molecular bridge 92. Controller 200 can further control the Fano resonance interrogating energy, the detection and/or the analysis of the signal obtained. In typical applications, the interrogation energy (expressed in electron volts (eV), for instance, can be within a range of from about −2 eV to about 2 eV.

The equipment and methods described herein can be further highlighted by a comparison with existing technologies.

While effective, there are many drawbacks to the SBS process, the most limiting being speed, instrumentation environmental requirements, reliance on fragile reagents, and computational complexity. With respect to speed, it can take days to weeks to sequence whole genomes depending on the organism. Illumina instruments, for instance, are benchtop or stand-alone tools that require conditioned lab environments to properly function and cold-chain access for storage of sequencing reagents which must be prepared (at least thermally conditioned) just prior to initiating a sequencing run. Finally, reconstructing fragmented sequences requires high-powered ASICs which, themselves, take days to compile a result for full genomes.

Approaches described herein can address many of these deficiencies. Techniques conducted according to embodiments of the invention, for example, can be used in multi-molecular sequencing and compare very favorably to other methods for characterizing DNA, e.g., in terms of error, speed, and scalability.

In many cases, error for DNA sequencing is the result of poor single base resolution resulting in miss-reads (wrong call, or missing call) and lack of distinction in homopolymer runs (strings of equivalent bases). The device, system and methods described herein can address these problems through highly distinct spectral identification of each base, an identification that involves multiple points of reference, not just a single scalar value such as conductance or ionic current.

In comparison to the leading next-generation technology, specifically as developed by Oxford Nanopore Technologies (ONT), practicing the invention can provide superior signal quality in its ability to unambiguously identify single bases, which is not possible with the Oxford Nanopore Technologies method, where the highest resolution is 5 bases and not single base, as in the present application. Embodiments of the invention provide a spectroscopically specific identifying signal for each nucleobase (or residue) of interest and thus also represent an improvement over other quantum-based technologies. Techniques developed by QuantumBioSystems, for example, rely exclusively on quantum conductivity not the transport spectrum. This is akin to identifying an organic compound by its bulk conductivity as opposed to its FTIR or NMR spectrum.

Furthermore, a high scan rate, in the range of 100 scans/base, is expected. This rate can allow detection of the onset, occupation, and exit of a base from the Fano resonator. Such high-resolution characterization of base transitions provides a means to easily distinguish bases within runs of equivalent residues (homopolymer runs).

Typically, the scan rate roughly sets the sequencing rate or speed. Even with a high scan rate of 100/base, a low frequency of 10 MHz would allow sequencing at a rate of 1×10⁵ bases/s which is much higher than ˜450 bases/s for Oxford Nanopore Technologies (ONT) nanopore, described by Lopez, R.; Chen, Y. J.; Dumas Ang, S.; Yekhanin, S.; Makarychev, K.; Racz, M. Z.; Seelig, G.; Strauss, K.; Ceze, L. DNA Assembly for Nanopore Data Storage Readout., Nat. Commun. 2019, 10, 1-9 or in Oxford-Nanopore-Releases-Rev-d-Flow-Cells-Enabling-Increase-Data-Yields @ Nanoporetech.com.

If scanning is increased to 1 GHz, sequencing could occur at 1×10⁷ bases/s which is even faster than unimpeded translocation through protein nanopores. (See, Wang, S.; Wang, Y.; Yan, S.; Du, X.; Zhang, P.; Chen, H.-Y.; Huang, S., Retarded Translocation of Nucleic Acids through α-Hemolysin Nanopore in the Presence of a Calcium Flux, ACS Appl. Mater. Interfaces 2020.

Devices working in parallel would allow even higher throughputs. This can be realized through the standard scalability of microfabricated devices. Even with challenges in realizing production scale throughput for fabricating bridged devices, early attempts have demonstrated reasonable yields, and techniques exist to easily identify missing or multiple bridging events. See, Sun, H.; Jiang, Z.; Xin, N.; Guo, X.; Hou, S.; Liao, J., Efficient Fabrication of Stable Graphene-Molecule-Graphene Single Molecule Junctions at Room Temperature, ChemPhysChem 2018, 19, 2258-2265; Wen, H.; Li, W.; Chen, J.; He, G.; Li, L.; Olson, M. A.; Sue, A. C. H.; Stoddart, J. F.; Guo, X., Complex Formation Dynamics in a Single Molecule Electronic Device, Sci. Adv. 2016, 2.

The invention also can find applications in fields other than DNA sequencing, to analyze biomolecules such as proteins or carbohydrates, where sequencing is much less systematic or consolidated.

Practicing embodiments of the invention can produce robust, standalone, miniaturized solutions with fast (e.g., minutes), real time reporting. With an anticipated footprint of 3000 μm² (e.g., 30 μm by 100 μm) for a nanochannel based system, QT-Seq devices would be small enough for deployment as remote sensors and robust enough to endure prolonged operation without the need for, or liability of, fragile biomolecular components. The rate of sequencing is expected to support real-time monitoring of transient DNA and potentially other molecules of interest.

Approaches described herein can find point of use applications in any number of fields, such as personalized medicine, counterfeiting investigations, viral genetic sensors, and so forth. Embodiments of the invention can have a major impact on any number of genetic and biotechnology fields such as CRISPR, bio informatics and personalized medicine, where consolidated sequencing of proteins would substantially accelerate development of bespoke protein and antibody-based therapeutics. For COVID19 and other viruses, practicing aspects of the invention offers the potential for highly deployed genetic sensors capable of detecting pathogens in a miniaturized (even hand-held) form factor, robust to environmental conditions, and self-contained needing nothing more than the sample itself for analysis (i.e. without need for fragile preparations components such as ratchet protein complexation used in ONP).

In addition to sequencing macromolecules, approaches described herein could be used in molecular informatics and in-line monitoring of small molecule solutions. For example, quantum transport analysis could be used to detect and analyze small molecules, e.g., in samples containing mixtures of small molecules, making possible in-line monitoring of molecular components with much higher specificity than FTIR, for instance. Furthermore, while LC-MS (liquid chromatography-mass spectrometry) can produce such analysis currently, LC-MS requires off-line operations, precluding real-time monitoring.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for fabricating a sequencing device, the method comprising:
applying a graphene monolayer onto a substrate to form a universal gate;
patterning the graphene monolayer into graphene nanoribbons separated by nanogaps;
depositing metal electrodes onto the graphene nanoribbons to form a FET array; and
depositing molecular bridges across the nanogaps,
wherein at least one of the molecular bridges has a cross section no greater than about 2 nanometers and a ballistic conductivity that is the same or substantially the same as the ballistic conductivity of a graphene nanoribbon.

2. The method according to claim 1, further comprising covering the graphene monolayer with a photoresist coating.

3. The method according to claim 1, further comprising shielding the electrodes with an insulating layer.

4. The method according to claim 1, wherein at least one of the nanogaps has a width no greater than about 5 nanometers.

5. The method according to claim 1, wherein the molecular bridges are prepared by DFT-guided solution-based synthesis.

6. The method according to claim 1, wherein at least one of the molecular bridges is configured for Fano resonance with a target molecule or a component thereof.

7. The method for of claim 1, wherein the sequencing device is integrated into a fluidic system for streaming a sample over the molecular bridges and/or wherein the sequencing device is integrated into a Fano resonance analysis system.

8. The method of claim 1, further comprising generating bridge molecule candidates, evaluating their Fano resonance transmission and synthesizing the molecular bridges using a selected candidate.

9. A sequencing device comprising:
a substrate supporting a graphene monolayer;
a molecular bridge at a nanogap defined in the graphene monolayer; and
electrodes for providing electron transport along the molecular bridge,
wherein the molecular bridge has a cross-section within a range of from about 1 to about 2 nanometers and has a ballistic conductivity that is the same or substantially the same as that of a nanographene ribbon.

10. The sequencing device according to claim 9, wherein the nanogap has a width no greater than about 5 nanometers.

11. The sequencing device according to claim 9, wherein bridge molecules are prepared by DFT-guided solution-based synthesis.

12. The sequencing device according to claim 9, further comprising an insulating layer shielding the electrodes.

13. The sequencing device according to claim 9, further comprising a photoresist coating over the graphene monolayer.

14. The sequencing device according to claim 9, wherein the molecular bridge is coupled to graphene electrodes.

15. The sequencing device according to claim 9, comprising an array including two or more molecular bridges.

16. The sequencing device according to claim 9, wherein the nanogap forms a fluidic channel for passing a sample across the molecular bridge.

17. The sequencing device according to claim 9, coupled to a controller for analyzing a sample by Fano resonance transmission.

18. The sequencing device according to claim 9, integrated into a fluidic system for streaming a sample over the molecular bridge.

19. A system sequencing system, comprising:
a flow chamber; and
a Fano resonator sequencing device comprising a molecular bridge at a nanogap defined in a graphene monolayer,
wherein the molecular bridge has a cross-section no greater than about 2 nanometers and has a ballistic conductivity that is the same or substantially the same as that of a nanographene ribbon.

20. The system according to claim 19, further comprising a controller.

21. The system according to claim 19, further comprising a sample processing component.

22. The system according to claim 19, further comprising a nano-slit and/or nanopillars for uncoiling a coiled macromolecule.

23. The system according to claim 19, wherein the molecular bridge is coupled to graphene electrodes.

24. A method for sequencing a molecule, the method comprising:
passing a sample comprising the molecule across a molecular bridge having a cross-section no greater than about 2 nanometers and a ballistic conductivity that is the same or substantially the same as the ballistic conductivity of a graphene nanoribbon; and
conducting a Fano resonance transmission analysis to identify the molecule or a component thereof.

25. The method according to claim 24, further comprising uncoiling a coiled molecule.

26. The method according to claim 24, wherein an electron transport is directed along the molecular bridge.

27. The method according to claim 24, wherein the molecule is a DNA molecule, a protein or a carbohydrate.

28. The method according to claim 24, further comprising developing scanning waveforms for analyzing the sample.
